(19) United States
(12) Patent Application Publication
(10) Pub. No.: US 2020/0345448 A1
(43) Pub. Date: Nov. 5, 2020

(54) CROSS POLARIZED SURGICAL LOUPES

(71) Applicant: Fireside Technologies LLC, Pikesville, MD (US)

(72) Inventor: Zain Ulabedin Syed, Pikesville, MD (US)

(21) Appl. No.: 16/865,546

(22) Filed: May 4, 2020

Related U.S. Application Data
(60) Provisional application No. 62/843,284, filed on May 3, 2019.

(51) Int. Cl. A61B 90/30; G02B 5/30; G01N 21/21

(52) U.S. Cl. CPC A61B 90/30; G02B 5/3025; A61B 2090/502; G01N 2201/0683; G01N 21/21

(57) ABSTRACT

A surgical loupe system includes one or more polarization filters for producing and/or blocking polarized light. A light source is directed through a polarization filter to a patient's tissues, producing returned light. The returned light may be magnified.

### RELATED APPLICATIONS

This application claims the benefit of U.S. Provisional Patent No. 62/843,284, filed on May 3, 2019, entitled “Cross Polarized Surgical Loupes” the contents of which are incorporated by reference herein.

### STATEMENT REGARDING FEDERALLY SPONSORED RESEARCH OR DEVELOPMENT

(Not Applicable)

### BACKGROUND

Surgical loupes are optical magnifying devices frequently used in surgeries to help the surgeon view small structures, which may benefit from the larger view. They are often attached to glasses, such as safety glasses, and may rotate into or out of the view field of the user. The introduction of a light source has been used in conjunction with surgical loupes to aid the user in viewing human skin surfaces and deeper tissue. Illumination from the light source may produce glare. In a surgical environment, the glare may obscure the patient's tissue, surgical instruments and other objects. The illumination from the light may be relatively intense to permit high visibility of the tissue, which may lead to constricted pupils for the surgeon, resulting in reduced visual acuity. In addition, the intense light and/or glare may lead to eye strain, fatigue and/or headaches, or other undesirable working conditions for the surgeon.

### SUMMARY

A cross polarized surgical loupe system is discussed herein. The disclosed system and method provides improved optics and advantages for surgeons using loupes.

In one example, dermatoscopic operations are improved. For example, improved identification of melanoma and other skin illnesses can be achieved.

In some examples, linearly polarized light is implemented with a light source, which may be a headlight, and polarizing filters. The linearly polarized light maintains polarization upon reflection.

In some examples, a polarized light filter is implemented with a surgical loupe to block light that is non-polarized, or polarized in a different orientation than the light filter. The polarized light filter may be arranged in conjunction with the spatial location of the polarized light from the light source. For example, the filters may be parallel or at an angle with respect to each other to achieve polarization and/or polarized filtering. In some example implementations, the angle of the filters is configured to permit polarized light from the filtered light source to be reflected back to the loupe lens.

In some example implementations, the orientation of the polarized filters are configured to permit polarized light from the filtered light source to be reflected back to the loupe lens. The arrangement and orientation of the polarization of the filters permits the loupe lens filter to block the reflected light from the patient derived from the incident polarized light from the filtered light source.

The scattering of light under a patient's skin or tissue surface may change the light's polarization. In some examples, a cross-polarized filter on or in conjunction with the loupe lens blocks light that is reflected directly from the light source, e.g., with no polarization change, and passes light with a polarization change, such as may be returned from under a patient's skin or tissue surface. The polarizers may be linear polarizers or radially (circular) polarized.

This cross-polarization technique improves loupe system operation. For example, vessel pattern, pigment network, color, and other subsurface feature visualization is improved. The light filter may reduce glare or light intensity to reduce eye strain and improve visual acuity. This glare may be reduced from both the patient's tissues as well as any instrumentation in the field of view.

### BRIEF DESCRIPTION OF THE SEVERAL VIEWS OF THE DRAWINGS

The disclosure is described in greater detail below, with reference to the accompanying drawings, in which:

FIG. 1 is a schematic view showing polarization of radiation;

FIG. 2 is a side perspective view of an example embodiment of the invention;

FIG. 3 is a side perspective view of an example embodiment of the invention;

FIG. 4 is a side perspective view of an example embodiment of the invention;

FIG. 5a is a front orthogonal view of an example embodiment of the invention;

FIG. 5b is a front orthogonal view of an example embodiment of the invention;

FIG. 6 is a flow diagram of an example method of the invention; and

FIG. 7 is a flow diagram of an example method of the invention.

### DETAILED DESCRIPTION

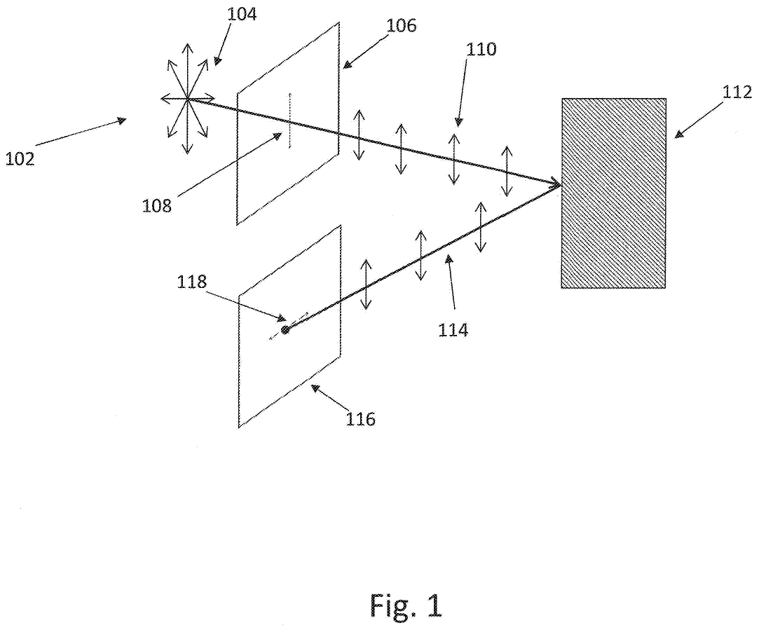

Referring to FIG. 1, polarizing filters may be used to block or pass radiation in a certain orientation. The orientation can be viewed as a phase of the radiation. A polarization filter for the source of radiation can be out of phase with the polarization filter for the returned radiation, whereby certain elements (orientations) of the radiation source are blocked in the returned radiation. As shown in FIG. 1, a source of electromagnetic radiation or transverse oscillating wave is shown as light source 102, having a ray of light with all possible orientations 104. The light source 102 is polarized by passing through a polarizing filter 106 which polarization axis is vertical, as shown by line segment 108, thereby creating vertical incident polarization of the electromagnetic radiation 110, prior to being applied to an object 112.

The returned electromagnetic radiation or transverse oscillating waves 114 are generated and reflected off the object 112. Portions of the returned (reflected and/or refracted) electromagnetic radiation or transverse oscillating wave may have a changed polarization from the incident polarization 110, by, for example, being scattered in orientation (not shown). As shown in FIG. 1, the returned and surface reflectance light 114 retains the vertical polarization orientation. The returned light 114 is again polarized by a second polarization filter 116 which has a horizontal orientation to its polarizing filter, shown by line segment 118, enabling the light which has a horizontal polarization axis to pass, producing a cross-polarized result. The cross-polarization permits some of the returned radiation to be blocked, resulting in a filtered signal that has desired characteristics.

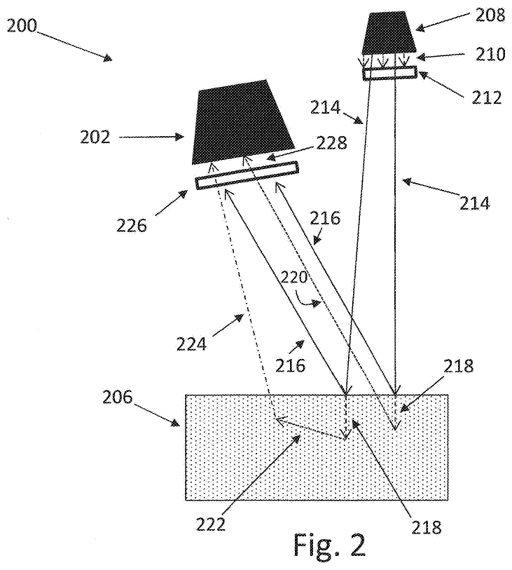

Referring to FIG. 2, there is shown an inspection system 200 described herein. Inspection system 200 is a multipurpose tool which may be used as a medical examination tool in these examples, the object of which is a tissue 206 of a patient. Inspection system 200 enables a medical practitioner to analyze and diagnose, among other diseases, hard to define skin cancers which have indistinct margins. As shown in FIG. 2, inspection system 200 includes a viewing lens 202. In this example, viewing lens 202 may be viewed by an eye of the user (not shown). The cross polarized image may also be viewed through viewing lens 202 by a visualizer, such as a camera, sensor, loupe device, etc.

Inspection system 200 as shown in FIG. 2 includes radiation in the form of a visible light source 208. LED light sources are frequently used, however any type of illumination that can be polarized may be used. The source in the general case may be any type of electromagnetic radiation or transverse oscillating wave that can be polarized. For example, light outside the visible spectrum, such as infrared or ultraviolet light may be used.

Referring to FIG. 2, light source 208 emits a light 210 that has differing angles of polarization. Light source 208 passes through a polarized light filter 212 which blocks or passes broad spectrum visible light used for illumination of tissue 206. Light 210 from light source 208 passes through polarized light filter 212, which has one polarization axis, to produce a polarized light 214. When polarized light 214 falls upon tissue 206, a portion of the light reflects off the surface of tissue 206 directly, as shown by light rays 216. Light rays 216 retain the polarization orientation of polarized light 214. Another portion of the incident light penetrates into tissue 206 as illustrated by light rays 218 and reflects back from a subsurface feature, as shown by the dotted line of a light ray 220. Other portions of incident light on tissue 206 bounces or changes direction multiple times before exiting tissue 206, as shown by the dotted line of a light ray 222 and a hashed line of a light ray 224. Light rays 218 and 222 shift their polarization orientation upon penetrating the tissue and upon every subsequent internal reflection or direction change. Light rays 220 and 224 have their polarization shifted slightly after internal reflection or direction change.

A polarized viewing filter 226 has a polarization axis which is 90 degrees, or orthogonal, to the polarization axis of polarized light filter 212. As light rays 216, 220 and 224 return from tissue 206, polarized viewing filter 226 filters out light rays 216 having the same polarization axis that was reflected from the light 214. Polarized viewing filter 226 allows or passes light rays 220 and 224 through the viewing filter 226 to produce light rays 228. Accordingly, polarized light filter 212 polarizes light 210 from light source 208 to produce polarized light 214 and polarized viewing filter 226 polarizes light rays 220 and 224 returned from tissue 206 to obtain a cross polarized image (not shown) of tissue 206 that is presented to viewing lens 202. Viewing lens 202 permits the cross polarized image to be viewed by an eye of a user (not shown), for example, or presented to any type of visualizer, such as a camera, sensor, loupe devices, etc.

The polarization filters may be any type of useful material, typically composed of glass or plastic. Glass filters may be more resistant to heat incurred from the light source. The polarization filters may be cooled via heat sinks or other cooling techniques, such as Peltier crystals. The polarization filters may be constructed as linear polarizers, circular polarizers, or other forms of polarizers, such as elliptical, for example. Wire polarizers may also be used where increased resistance to heat is desired. In an example using linear polarizers, as shown in FIG. 2, the filters are arranged to be normal or orthogonal to the light source direction. However, any type of cross polarization arrangement may be used. For example, in each of the embodiments discussed herein, orthogonal cross polarization is mentioned, however any angle or phasing of cross polarization may be used, such as in a range of from zero to ninety degrees. The filters may be arranged to be parallel or non-parallel with each other, and may be subjected to highly precise relative orientation with the light source and each other to increase the efficiency of polarization. The filters may be adjustable to modify the cross polarization, for example by changing the angle of polarization of one or more of the filters.

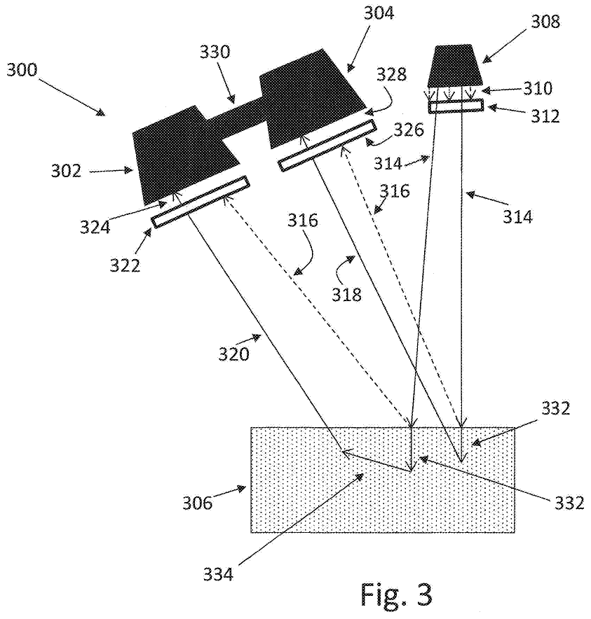

In an example using circular polarizers, as shown in FIG. 3, the filters may be arranged to be parallel to the light source direction. The filters are arranged to be parallel with each other. The circular polarizers may have a less precise orientation relative to the light source and each other. The circular polarizers do not require a highly accurate angle with the light source direction. In an example embodiment, linear polarizers could also be utilized with the viewing lenses and light source.

As shown in FIG. 3, the inspection system 300 includes two viewing lenses including a first viewing lens 302 and a second viewing lens 304 configured on a parallel support means 330, thereby enabling binocular viewing of tissue 306. The first viewing lens 302 and second viewing lens 304 may also be detached from each other. Such viewing may be accomplished by eyes of a user (not shown) or the cross polarized image may also be viewed by a visualizer, such as a camera, sensor, loupe devices, etc.

The inspection system as shown in FIG. 3 includes radiation in the form of visible light 308. In view of the circular polarizers not requiring a highly accurate angle with the light source direction, the light source 308 may be configured on a separate device than the first viewing lens 302 and second viewing lens 304, as shown in FIG. 3. Alternatively, the light source 308 may be adapted to be secured to the first viewing lens 302 and second viewing lens 304. As shown in FIG. 3, light source 308 emits a light 310 that is polarized in multiple different ways, as shown by hashed/dotted lines and straight lines representing differing angles of polarization. Once the light 310 passes through the first circular polarizer 312, only one handedness of light is permitted through, to produce a polarized light 314. When the polarized light 314 falls upon the object being viewed, which is tissue 306 in FIG. 3, a portion of the light reflects off the surface of tissue 306 directly and reverses the polarization handedness, as shown by light rays 316, while the remaining light rays penetrate into the object, and reflects back from a subsurface feature, as illustrated by light rays 332, or bounces or changes direction multiple times, as shown by light rays 334, before exiting tissue 306, as shown by light rays 318 and 320, respectively. Light rays 318 and 320 have their polarization shifted slightly after internal reflection or direction change, but do not reverse their overall handedness. A second circular polarizer 322 has a handedness that is equivalent to the handedness of the first circular polarizer 312, and only allows similarly handed light rays 324 to pass through, while blocking the reflected lights rays 316 that have fully reversed handedness. A third circular polarizer 326 has a handedness that is equivalent to the handedness of the first circular polarizer 312 and handedness of the second circular polarizer 322 and allows similarly handed light rays 328 to pass through, while blocking the reflected light rays 316 that have fully reversed handedness. Light rays 324 pass through the second circular polarizer 322 and light rays 328 pass through the third circular polarizer 326 to obtain a cross polarized image (not shown) in the first viewing lens 302 and the second viewing lens 304, respectively. First viewing lens 302 and second viewing lens 304 permit the cross polarized image to be viewed by an eye of a user (not shown), for example, or presented to any type of visualizer, such as a camera, sensor, loupe devices, etc. Alternatively, in an example embodiment, light source 308 may also be configured on a device including only a first viewing lens 302 and be viewed by only one eye of a user or one visualizer.

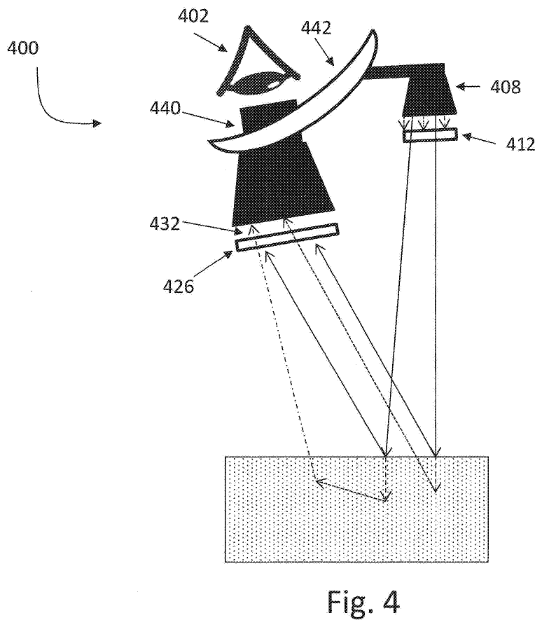

The inspection system as shown in FIGS. 2 and 3 may also be configured as part of a portable head device worn by medical practitioners allowing them to remain hands free during an exam of a patient. The light source may be attached to the head device, as shown in FIG. 4, or may be configured as a separate device, as shown in FIGS. 2 and 3. In an example embodiment, the light source may be integrally molded to the first and second viewing lenses of the inspection system.

Referring to FIG. 4, the inspection system 400 may include a surgical loupe 440 to magnify an image. The resulting magnified filtered light provides the user with greater visual acuity and images to contribute to improved diagnoses and observations. Some challenges to using surgical loupes include the glare produced with a broad spectrum light source used for illumination of the tissue. The glare may obscure the tissue, surgical instruments and other objects in a surgical environment. The illumination may be relatively intense to permit high visibility of the tissue, which may lead to constricted pupils for the surgeon, resulting in reduced visual acuity. For example, visual acuity may be reduced when pupils become smaller than 2 mm. In addition, the intense light and/or glare may lead to eye strain, fatigue and/or headaches, or other undesirable working conditions for the surgeon. Moreover, illumination is often provided by high power LEDs, which emit a significant amount of high intensity blue light, which has been linked to macular degeneration. The inspection system of the present invention may be used by others working under conditions needing similar glare reduction, such as those working in the jewelry and watch manufacturing industries. Polarized loupes are available, but do not significantly reduce glare. Rather, these polarized loupes tend to reduce the light intensity of the reflected light.

Referring to FIG. 4, magnification of the returned light 432 is shown. A loupe device 440 is disposed between the eye of the user 402 and the polarized viewing filter 426. A light source 408 may be adapted to be secured to loupe device 440 through connector 442. The loupe device 440 receives the cross polarized light 432 which has passed through the polarized viewing filter 426 having a polarization axis orthogonal to the polarized light filter 412, thereby creating a polarized image before being observed, detected or captured by a visualizer, such as an eye of the user 402. The magnified cross polarized image may also be presented to a detector or sensor, such as an image capture device or camera.

When the light source is polarized, and the returned light from an object is also polarized, out of phase with the source polarized light, the glare seen by the visualizer is nearly or completely eliminated. This cross polarization of the returned light permits some features below the surface of the tissue to be observed more clearly. Wet objects, rather than appearing intensely bright due to glare, appear to be dry. Metallic objects, rather than reflecting incident light back to the user's eyes, appear in their natural color without glare. Thus, the cross polarized system avoids glare. This result can be seen even when very bright light (polarized) is directed directly at the loupe device.

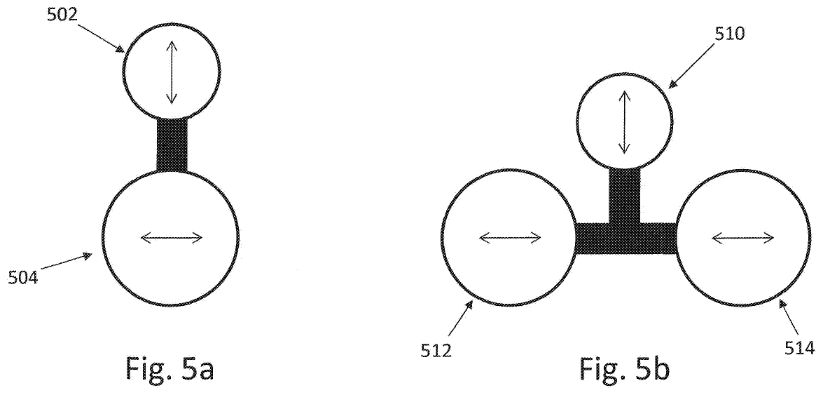

Referring to FIG. 5a, a front orthogonal view of an example embodiment is illustrated with polarized light filter 502 and polarized viewing filter 504, using linear polarizers. The polarized light filter 502 and polarized viewing filter 504 may be configured with circular polarizers, wherein the polarized light filter 502 and the polarized viewing filter 504 would have the same polarization handedness. As shown in FIG. 5a, the polarized light filter 502, disposed in front of a headlight (not shown), has a vertical polarization axis. The polarized viewing filter 504, disposed in front of visualizers, which may include but not be limited to loupes, magnifiers, cameras, sensors, etc. (not shown), has an orthogonal polarization axis, i.e., horizontal, with respect to the polarized light filter 502. In this example embodiment, the visualizer (not shown) may be configured to be attached to the polarized viewing filter 504. In an alternative embodiment, the visualizer (not shown) may be configured to be separate and disposed adjacent to the polarized viewing filter 504.

Referring to FIG. 5b, a front orthogonal view of an example embodiment is illustrated with polarized light filter 510 and polarized viewing filters 512 and 514, using linear polarizers. The polarized light filter 510 and polarized viewing filters 512 and 514 may also be configured with circular polarizers, wherein the polarized light filter 512 and polarized viewing filters 512 and 514 would all have the same polarization handedness. As shown in FIG. 5b, the polarized light filter 512, disposed in front of a headlight (not shown), has a vertical polarization axis. In this example embodiment, the polarized light filter 512 is disposed adjacent to and between polarized viewing filters 512 and 514. The polarized viewing filters 512 and 514, disposed in front of visualizers, which may include but not be limited to loupes, magnifiers, cameras, sensors, etc. (not shown), each have an orthogonal polarization axis, i.e., horizontal, with respect to the polarized light filter 512. In an example embodiment, the visualizer (not shown) may be configured to be attached to the polarized viewing filter. In an example embodiment, the visualizer is comprised of magnification loupes, and provides the user with binocular loupes for viewing an object. In an example embodiment, the binocular loupes (not shown) are configured adjacent to each other and fixedly attached to each other and to the headlight (not shown). In an example embodiment, the binocular loupes (not shown) are configured parallel and attached to the polarized viewing filters 512 and 514 and the headlight (not shown) is configured parallel and attached to the polarized light filter 510. In an alternative embodiment, the visualizers (not shown) may be configured to be separate and parallel to the respective polarized viewing filters 512 and 514, and the headlight (not shown) may be configured to be separate and parallel to the polarized light filter 510. In yet another alternative embodiment, there may be additional polarized viewing filters and associated visualizers disposed on the inspection device.

Referring to FIG. 5b, with the use of linear polarizers, the two polarized viewing filters 512 and 514 are configured to be in the equivalent orientation and alignment with each other. This may be accomplished by, among other means, aligning the two polarized viewing filters 512 and 514 such that they are similarly oriented to each other and orthogonally oriented with respect to the polarized light filter 510. This may be accomplished with the use of an external reflective source (not shown) adapted to reflect light back onto the polarized viewing filters 512 and 514. In particular, the polarized viewing filters 512 and 514 may be configured as part of a portable head device enabling a user to remain hands free. The light source may be attached to or integral with the head device or may be configured as a separate device. One or more polarized viewing filters may be attached to a loupe device (not shown). The user would turn on the polarized light source and look through the loupe device while rotating the polarized viewing filter 512 in front of the loupe device. The greatest amount of light passes through when the polarized viewing filter is aligned in the same polarization axis as the polarized light source. As the user further rotates the polarized viewing filter to its horizontal axis, the least amount of light passes through the polarized viewing filter. At such point, the same process is performed for the other polarized viewing filter 514 for the second visualizer. When the darkest point occurs with respect to each polarized viewing filter, the inspection system is configured to obtain a cross polarized image.

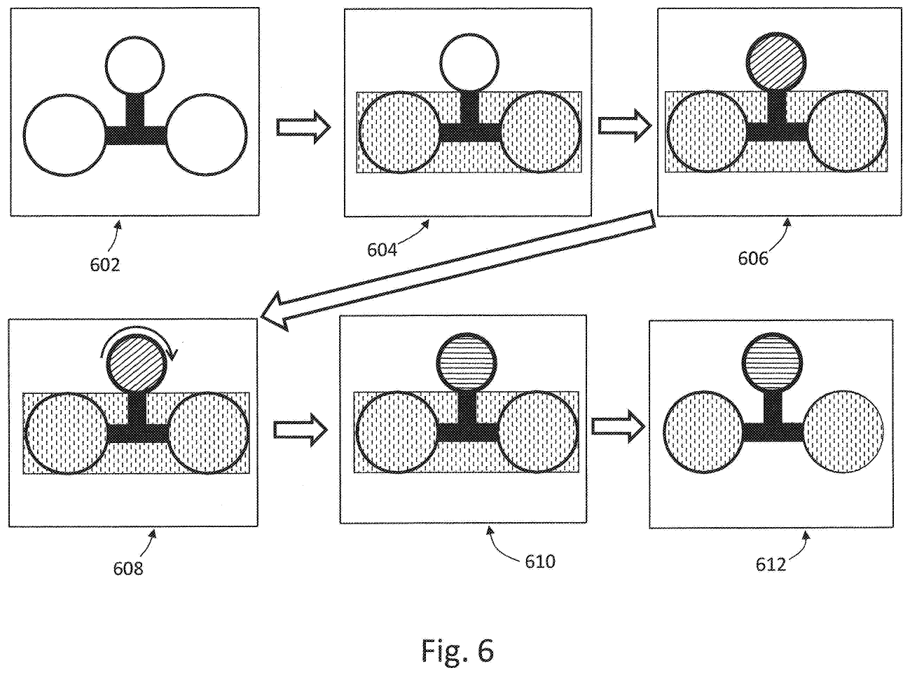

Referring to FIG. 6, there is shown an example method for constructing cross polarized first and second viewing lenses. First and second viewing lenses may be constructed by securing the viewing lenses parallel to each other by means of a shaft or other mounting device and mounting the light source to the mounting device, as shown in step 602. Polarizing film is applied across viewing devices through the use of adhesives or other similarly adhering means, as shown in step 604. Polarizing sheet or film may be constructed from plastic or glass. The polarizing sheet or film may be linearly polarized or radially (circular) polarized. FIG. 6 shows two polarized viewing filters configured to be in the equivalent orientation and alignment with each other, as shown by vertical hash lines in step 604. The polarizing sheet ensures similar polarization axes for both polarized viewing filters. As shown by the vertical hashed lines in the polarizing sheet in step 604, the orientation of polarization of the polarizing sheet is vertical. The polarizing sheet may have any orientation. A polarized light filter, which may be constructed of glass, is attached to the light source, as shown in step 606. The orientation of the polarized light filter is shown by diagonal hashed lines in step 606. While viewing a mirrored surface (mirror or metal) (not shown) through the first and second viewing lenses, which may be performed by a user or other visualizer device such as a camera or sensor device, the reflection of the headlight is viewed through the mirrored surface. As the polarized light filter is rotated, as shown in step 608, the light from the light source will diminish in intensity until it reaches a minimum point, and then start to get brighter again. When the light is at its minimum, the polarized light filter is locked into place, as shown in step 610. At such point the polarization axis of the polarized light filter is orthogonal to the polarization axis of both first and second polarized viewing filters, as shown by the hashed lines in step 610. This represents full cross polarization. After locking light filter, a user or other visualizer device may confirm such cross polarization by looking through the first and second polarized viewing filter. The polarizing film may then be cut to remove excess film, as shown in step 612, such that the remaining film fully covers the polarized viewing filters.

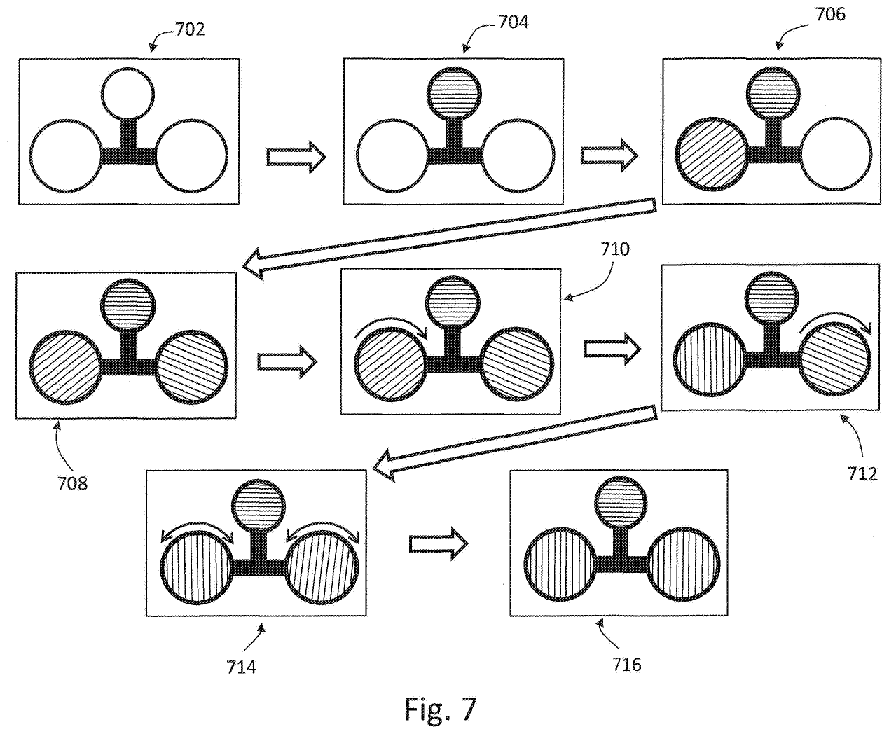

Referring to FIG. 7, another example method is shown for cross polarization. As shown in FIG. 7, first and second polarized viewing filters and polarized light filter may be constructed by securing the first and second viewing lenses parallel to each other by means of a shaft or other mounting device and mounting the light source to the mounting device, as shown in step 702. The polarized viewing filters and polarized light filter may be linearly polarized or radially (circular) polarized. The polarized light filter, which may be configured from a discrete sheet of glass or plastic, is attached to the light source and locked into place, as shown in step 704. The horizontal orientation of the polarized light filter is shown in horizontal hashed lines in step 704. The polarized light filter may have any orientation.

The first polarized viewing filter is attached to the first viewing lens, as shown in step 706. The second polarized viewing filter is attached to the second viewing lens, as shown in step 708. The orientation of the first polarized viewing filter and second polarized viewing filter may be the same or out of phase with each other or with the polarized light filter, as shown by the hashed lines of the first and second polarized viewing filter and polarized light filter, in step 708. While viewing a mirrored surface (mirror, metal, or other reflective surface) (not shown) through the first and second polarized viewing filters, the reflection of the light source is shown, and the first polarized viewing filter is rotated, as shown in step 710. The light will diminish in intensity until it reaches a minimum point, while viewed through the first polarized viewing filter. The brightness of the light source while viewed from the second polarized viewing filter will remain constant, and then start to get brighter again during such rotation of the first polarized viewing filter. When the light is at its minimum, the rotation of the first polarized viewing filter is halted, as shown in step 712. This represents full cross polarization of the first polarized viewing filter. This process is repeated for the second polarized viewing filter, as shown in step 712. Further rotation of the first and second polarized viewing filters may be performed to fine tune both first and second polarized viewing filters, as shown in step 714 until the intensity of the light through both lenses is at a minimum and the reflected light matches color and intensity as closely as possible. Once this has been achieved, both polarized viewing filter 712 and 714 are locked into place, as shown in step 716, thereby creating a cross polarized image, as further shown by the vertical orientation of hashed lines in the first and second polarized viewing filters and the horizontal hashed lines in the polarized light filter.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

What is claimed is:

1. A cross polarized inspection system, comprising:
a source of electromagnetic radiation for irradiating an object;
a first polarization filter between the object and the source;
a second polarization filter arranged to receive returned electromagnetic radiation from the object; and
the first polarization filter and the second polarization filter being out of phase with each other.

2. The system of claim 1, wherein the electromagnetic radiation is visible light.

3. The system of claim 1, further comprising
the first polarization filter including a first polarization axis;
the second polarization filter including a second polarization axis; and
wherein the first polarization axis is orthogonal to the second polarization axis.

4. The system of claim 3, further comprising a first optical magnifier on one side of the second polarization filter.

5. The system of claim 4, wherein the source is secured to the first optical magnifier.

6. The system of claim 4, further comprising
a third polarization filter arranged to receive returned electromagnetic radiation from the object; and a second optical magnifier on one side of the third polarization filter.

7. The system of claim 6, wherein the first optical magnifier is secured to the second optical magnifier.

8. The system of claim 7, wherein the source is secured to the first optical magnifier and second optical magnifier.

9. The system of claim 1, wherein the polarization filters are linear polarizers.

10. The system of claim 1, further comprising
the first polarization filter including a first polarization handedness;
the second polarization filter including a second polarization handedness; and
wherein the first polarization handedness is equivalent to the second polarization handedness.

11. The system of claim 10, further comprising a first optical magnifier on one side of the second polarization filter.

12. The system of claim 11, wherein the source is secured to the first optical magnifier.

13. The system of claim 11, further comprising
a third polarization filter arranged to receive returned electromagnetic radiation from the object; and a second optical magnifier on one side of the third polarization filter.

14. The system of claim 13, wherein the first optical magnifier is secured to the second optical magnifier.

15. The system of claim 14, wherein the source is secured to the first optical magnifier and second optical magnifier.

16. The system of claim 10, wherein the polarization filters are circular polarizers.

17. A method for inspecting an object, comprising:
providing a source of electromagnetic radiation for irradiating an object;
providing a first polarization filter between the object and the source;
providing a second polarization filter arranged to receive returned electromagnetic radiation from the object; and
arranging the first polarization filter and the second polarization filter to be out of phase with each other.

18. A method for inspecting an object, comprising:
providing a source of electromagnetic radiation for irradiating an object;
providing a first polarization filter between the object and the source;
providing a second polarization filter arranged to receive returned electromagnetic radiation from the object; and
arranging the first polarization filter and the second polarization filter to have the same handedness.

19. A method for inspecting an object, comprising:
applying polarized electromagnetic radiation to the object to produce returned polarized radiation; and
cross polarizing the returned polarized radiation.

20. The method of claim 19, further comprising applying a first optical magnifier to the returned polarized radiation.

21. The method of claim 20, further comprising applying a second optical magnifier to the returned polarized radiation.

22. The method of claim 20, wherein the cross polarizing of the returned polarized radiation is formed by utilizing a polarization film.

23. The method of claim 20, wherein the cross polarizing of the returned polarized radiation is formed by sequentially rotating parallel polarized viewing filters.

24. The method of claim 22, wherein the polarization film is linearly polarized.

25. The method of claim 22, wherein the polarization film is radially polarized.
